(19) United States
(12) Patent Application Publication
(10) Pub. No.: US 2009/0030424 A1
(43) Pub. Date: Jan. 29, 2009

(54) Surgical device for folding objects and methods of using the same

(75) Inventors: Sonal S. Tuli, Gainesville, FL (US); Donald M. Downer, Gainesville, FL (US)

Correspondence Address: EDWARDS ANGELL PALMER & DODGE LLP, P.O. BOX 55874, BOSTON, MA, 02205, US

(73) Assignee: University of Florida Research Foundation

(21) Appl. No.: 11/883,711

(22) PCT Filed: Feb. 2, 2006
(86) PCT No.: PCT/US06/03699

§ 371 Date: Apr. 15, 2008

Related U.S. Application Data
(60) Provisional application No. 60/649,381, filed on Feb. 2, 2005.

(51) Int. Cl. A61F 9/007; A61B 17/28

(52) U.S. Cl. 606/107; 606/205

(57) ABSTRACT

Disclosed are new and useful three-prong forceps devices for grasping and folding objects such as implanted surgical devices, and methods of use thereof. Certain embodiments of the devices are configured for folding and explanting an intraocular lens from the anterior chamber of the eye of a patient through a small incision in the cornea, reducing trauma to the eye.

### CROSS-REFERENCE TO RELATED APPLICATIONS

The present application claims the priority benefit of U.S. Provisional Patent Application Ser. No. 60/649,381 entitled “Intraocular Lens Explantation Devices and Methods of Using the Same,” filed on Feb. 2, 2005, the disclosure of which is hereby incorporated by reference in its entirety.

### BACKGROUND OF THE INVENTION

1. Field of the Invention

The subject invention relates to the general field of surgery, and more particularly to surgical procedures and tools designed to minimize trauma to tissues. Disclosed are devices and methods for folding objects to be removed from a surgical site through an opening, such as an incision, that is smaller than the object to be removed. Particular embodiments are useful for removal of a foldable intraocular lens (IOL) from a patient's eye.

2. Background of the Related Art

It is recognized in the medical arts that there are many benefits to minimizing the trauma to a surgical site. In many areas of surgery, new techniques involving minimally invasive procedures are in favor, providing the benefits of faster healing, reduced intraoperative time and patient stays in medical facilities.

The field of opthalmology is no exception to this trend. The human eye is a sensory organ which is essential to our perception of the world around us. The sense of sight is considered one of the most complex of the five senses and it requires the proper functioning and collaboration of several elements within the eye if clear vision is to exist. Unfortunately, factors such as disease, age and injury can often affect one or more of these elements, causing visual capacity to significantly decrease.

The intraocular lens (IOL) was developed to replace the existing crystalline lens in the eye when the natural lens becomes clouded by the presence of a cataract or when the lens is unable to sharply focus light rays on the retina. A typical IOL comprises an optic (lens portion) which is approximately 6.0 mm wide and 0.5 mm thick, with attached fixation members, also termed “haptics,” oriented for example in a modified J configuration. Haptics are used to keep the lens in place and to prevent rotation after insertion into the lens capsule of the eye.

As in other areas of medicine, in recent years, surgical procedures have been developed to reduce trauma to the eye during ophthalmic surgery. In the area of cataract surgery, the past decade has brought a gradual shift toward smaller incisions (e.g., 3.0 to 3.5 mm wide) for the implantation of IOLs. The shift is largely the result of development of foldable IOLs, as well as techniques and tools for injecting folded IOLs into the eye through the smaller incisions.

A functioning natural lens can change shape to adjust for close or distance vision, in a process known as accommodation. The most commonly implanted IOLs can perfect either near or farsightedness, but not both. However, recent technologies have facilitated the development of new IOLs with accommodative abilities. Patients who receive the accomodative type of lens can see both near and at a distance without the use of glasses, whereas patients with older IOLs most often depend on glasses to see at all distance ranges. Therefore, in some instances it is advantageous to replace the existing IOLs with the new, accommodative IOLs.

Implanted intraocular lenses routinely must be extracted for many reasons, including but not limited to incorrect power calculation, intolerance of a multifocal IOL, IOL damage during insertion, intraoperative and postoperative IOL displacement, or replacement with accomodative lenses, as discussed above.

Historically, the explantation or removal of foldable lenses required substantial enlargement of the original 3.0 mm incision/wound or the creation of a new incision. More specifically, in order to remove the lens, an incision size of 4.0 mm or larger was made in order to facilitate the insertion of intraocular scissors which are used to sever the lens into two pieces. Once the lens is severed, each of the individual pieces is removed.

The enlargement of the original wound and/or the creation of a second wound site increases the risk of infection and wound leakage and also increases the risk of postoperative inflammation and greater astigmatism.

More recently, techniques have been developed for explanting an IOL through the original 3.0 mm incision used for insertion of the lens. These techniques also involve bisecting the IOL using scissors or a device that includes a wire sling. In such techniques, if the lens is not cut perfectly, there exists a risk of retaining lens fragments in the eye. Moreover, the methods are performed in multiple steps, which increases the duration of the surgical procedure and trauma to the eye.

Typically, intraocular lenses are made of acrylic or silicone materials. Acrylic materials are quite stiff at room temperature, but are soft and flexible at body temperature. Thus, although these lenses are difficult to fold in the operating room prior to insertion, once the lens is warmed, they can be easily and gently folded. On the other hand, silicone IOLs are easier to fold in the operating room at room temperature if they are dry (i.e., not covered by fluid). Once in the eye, silicone IOLs are slippery, resisting manipulation and folding attempts.

A technique has been developed for the intraocular folding of an acrylic lens for explantation through a small incision. (See Neuhann T., Intraocular folding of an acrylic lens for explantation through a small incision cataract wound, J. Cataract Refract. Surg. 1996; 22:1383-1386.) One disadvantage of this technique is that it is limited to acrylic IOLs, due to the slippery nature of silicone IOLs when implanted. A further, more significant disadvantage of this technique is that it is time consuming, complex and uses instruments that can cause trauma to the eye during IOL removal. The Neuhann procedure removes the IOL with a spatula and folding forceps. After the lens is rotated out of the capsular bag into the anterior chamber, the spatula is brought into the chamber underneath the IOL optic. Using the folding forceps, which are introduced through the limbal incision, the acrylic optic is bent carefully over the spatula. Then the captured spatula must be freed and the forceps with the folded IOL must be rotated 90 degrees. This folding technique is complex, time consuming and can result in surgical trauma to the eye, due to the need to manipulate the spatula and the forceps while they are positioned within the anterior chamber of the eye.

In many other areas of medicine, there is a need to surgically remove foreign or therapeutic or cosmetic devices that are implanted in the body. In most cases, it is desirable to remove the foreign object through the smallest incision possible. Therefore, there is a need for a device and method for removing a foreign object from the body, such as explanting an intraocular lens from a patient's eye as described above, without the need to enlarge the original incision. Still further, in particular ophthalmic applications, there is a need for methods and devices that provide a simple technique for folding IOLs within the anterior chamber of the eye and reducing the potential for trauma to the eye during IOL explantation.

### SUMMARY OF THE INVENTION

The present invention relates to devices, and methods for using the same, that are useful for removing (explanting) objects such as certain foldable medical devices and implants from a patient's body. The subject devices and methods advantageously permit explantation of such objects in a manner that limits tissue damage and reduces healing time by facilitating the use of incisions that are smaller than the objects to be removed. Such use of smaller incisions is made possible by imparting folds or curls in the objects prior to their removal through an opening such as an incision in the skin, or cornea, for example.

One preferred aspect of the invention is a forceps device for folding an object. In one embodiment, the device includes a first scissor arm having a first prong formed at an end thereof and a handle formed at a second end thereof, and a second scissor arm having first and second spaced apart prongs formed at an end thereof and including a handle formed at a second end thereof. The second scissor arm is rotatably mounted to the first scissor arm. In the open position, the handle portions of the first and second scissor arms are spaced apart, and the first prong formed on the first scissor arm is positioned below the first and second prongs of the second scissor arm. In the closed position, the spacing between the handle portions of the first and second scissor arms is reduced and the first prong formed on the first scissor arm is positioned above the first and second prongs of the second scissor arm. In the operation of the scissor-arm forceps, a fold is imparted in an object contacted by the prongs during the movement of the prongs from the open position to the closed position.

Configured according to this embodiment, the subject scissor arm forceps can be used alone, or in conjunction with an explantation device as further described below.

A second preferred embodiment of the invention is a retractable forceps device for folding and containing an object. The forceps device includes a tubular body member having a proximal end and a distal end and an inner surface extending therebetween. As used herein, the “proximal end” of a device or object is the end closest in proximity to the user (such as a surgeon) and the “distal end” of an object refers to the end closest in proximity to the object to be grasped. As used herein, a “tubular” configuration is not intended to be limited to those elements having a round or elliptical cross-section, but includes elements that have a hollow passage extending axially through the body of the element. In alternative embodiments, the cross-section of the main body can be, for example, circular, rectangular or a square. Typically, in embodiments having a rectangular or square cross-section, the corners of the internal passage are rounded.

Grasping of objects is accomplished by means of a first arm having a first prong formed at an end thereof and a second arm having first and second spaced-part prongs formed at an end thereof. A handle is formed at a second end of the arms. The first and second arms and prongs are operatively connected to the handle and are retractable into the tubular body member by pulling on the handle.

In the open position, the prongs are extended distal to the tubular body member, and the first prong formed on the first arm is positioned below the first and second prongs of the second arm. In the closed position, at least the proximal portion of the prongs is retracted into the tubular body member and the first prong formed on the first arm is positioned above the first and second prongs of the second arm, and the angle between the first and second prongs on the second arm is reduced. A fold or curl is imparted in an object contacted by the prongs during movement of the prongs from the opened to the closed position. In this embodiment, the prong-closing movement is effectuated by retracting the prongs into the tubular body member by means of the handle. As used herein, the terms “above” and “below” are merely relative terms, used to describe the relative positions of the prongs of a forceps device to one another as the device is held in one position (i.e., without rotating the device along its long axis) and the prongs are opened and closed. The devices may be held, and objects may be grasped in any orientation, and accordingly depending upon the orientation, a particular prong or pair of prongs in the 3-prong device may be either “above” or “below” an object that is grasped.

Certain embodiments of the retractable forceps device are configured for use in a particular surgical application, such as explanting an intraocular lens from a patient's eye. A retractable forceps device designed for this purpose includes a tubular main body having a proximal end and a distal end and an inner surface extending therebetween, with the inner surface defining an interior lens-receiving chamber adapted and configured for receiving an intraocular lens.

In some embodiments of the retractable forceps useful for lens explantation, the cross-section of the tubular main body taken in a plane which is orthogonal to the central axis is elliptical. In a particularly preferred embodiment, the elliptical cross-section for the tubular main body has a major axis of about 2.7 mm and a minor axis of about 1.65 mm.

Another aspect of the invention is a device for folding and containing an object, which includes the scissors-type forceps device as described above, and a separate tubular device for guiding the forceps and containing the folded object. The configuration of the forceps and tubular devices varies according to the nature of the object to be folded and contained.

Certain preferred embodiments of this device are configured for use in explanting an intraocular lens from a patient's eye. In these embodiments, the tubular portion of the device, termed a “lens explantation device” comprises a tubular main body having a proximal end and a distal end and an inner surface extending therebetween. The inner surface defines an interior lens-receiving chamber and a central axis for the explantation device. In a preferred embodiment of the explantation device, the cross-section of the tubular main body, taken in a plane that is orthogonal to the central axis, is elliptical. In certain embodiments in accordance with this shape, the elliptical cross-section for the tubular main body has a major axis of about 2.7 mm and a minor axis of about 1.65 mm.

In various embodiments, the inner surface of the tubular main body of the explantation device has certain modifications that promote folding of an object (such as a lens) as it is drawn through the lens-receiving chamber.

A representative embodiment of the lens explantation device of the present disclosure can further include a fin element that is attached to an inner surface of the tubular main body and projects inward from the inner surface into the interior lens receiving chamber. Preferably, the fin element is attached to the inner surface of the tubular main body at a location offset from the major axis of the tubular main body. In a representative construction, the fin projects inward from between about 0.25 to about 0.5 mm from the inner surface of the tubular main body. The fin element, rather than having a substantially rectangular cross-section, can have a smooth cross-section similar to a triangle.

Alternative embodiments of the present invention have a groove or channel formed in the inner surface of the tubular main body in lieu of a fin element. The location of the groove or channel can be similar to that described for the fin element above.

The lens explantation device of the present disclosure can further include a stop flange formed on the proximal end of the tubular main body for preventing the explantation device from being inserted too deeply into a patient's eye.

In another aspect, the present invention is directed to a method for explanting an intraocular lens from the anterior chamber of a patient's eye. One representative method of the present invention uses a retractable forceps as described above, and can be summarized as follows.

First, the distal end of the tubular body member of a retractable forceps device is inserted into a wound formed in the patient's cornea. The prongs of the forceps device are advanced in an open position beyond the distal end of the tubular body member into the anterior chamber of the eye. Next, the prongs of the retractable forceps are used to grasp and fold the intraocular lens. The forceps are retracted, thereby withdrawing the grasped and folded lens into the interior lens-receiving chamber. Upon completion of this step, the retractable forceps device having the intraocular lens disposed within the interior lens-receiving chamber is removed from the patient's eye.

Another representative method for explanting an intraocular lens uses a grasping means such as a scissors-type forceps for folding an object and a separate lens explantation device, as previously described. This method can be summarized as follows. First the distal end of a lens explantation device of the present invention is inserted into wound or incision formed in the patient's cornea. Then a device, such as a forceps for example, is inserted into the interior lens-receiving chamber so that it extends from the distal end of the tubular main body into the anterior chamber of the eye. The forceps are used to grasp the intraocular lens and to impart an initial fold in the lens. Then the lens is retracted into the interior lens-receiving chamber of the explantation device. Initially, in certain embodiments, the lens contacts the groove, channel, or fin element and a portion of the inner wall of the tubular main body and is partially restrained thereby. The lens is further retracted in the proximal direction such that the lens is folded thereby. Lastly, the explantation device which has the intraocular lens disposed within the interior lens receiving chamber is removed from the patient's eye.

These and other unique features of the device and method disclosed herein will become more readily apparent from the following description and the accompanying drawings.

### BRIEF DESCRIPTION OF THE DRAWINGS

So that those having ordinary skill in the art to which the disclosure appertains will more readily understand how to make and use the same, reference may be made to the drawings wherein:

FIGS. 1A-C are views of an embodiment of a scissor arm three-prong forcep device for folding an object, in accordance with the present invention. FIG. 1A is a perspective view seen from the side and FIGS. 1B and 1C are end-on views, illustrating the operation of the device.

FIGS. 2A-B are two perspective views of a retractable forceps device of the invention, viewed from the side (2A) and from below (2B).

FIGS. 3A-C are two perspective views (top, side), and end-on view, respectively, illustrating an initial stage of grasping an object by an embodiment of a retractable forceps device of the present invention.

FIGS. 4A-E are five views (A, top; B, D, side; and C, E, end-on) illustrating later stages of grasping and folding an object by an embodiment of a retractable forceps device of the present invention.

FIGS. 5A-C are three perspective views, shown from above, illustrating steps in the folding and removal of an intraocular lens from the anterior chamber of a patient's eye using a retractable forceps device in accordance with the invention.

FIGS. 6A-C are three perspective views, showing corresponding side views of the procedure to remove an intraocular lens illustrated in FIGS. 5A-C.

FIG. 7 is a perspective view of an embodiment of an explantation device in accordance with the invention.

FIG. 8 is a perspective view of an alternate embodiment of an explantation device in accordance with the invention.

FIG. 9 is a perspective view of yet another alternate embodiment of an explantation device in accordance with the invention.

### DETAILED DESCRIPTION OF PREFERRED EMBODIMENTS

Reference is now made to the accompanying figures for the purpose of describing, in detail, preferred and exemplary embodiments of the present disclosure. The figures and accompanying detailed description are provided to describe and illustrate exemplary manners in which the disclosed subject matter may be made and used, and are not intended to limit the scope thereof.

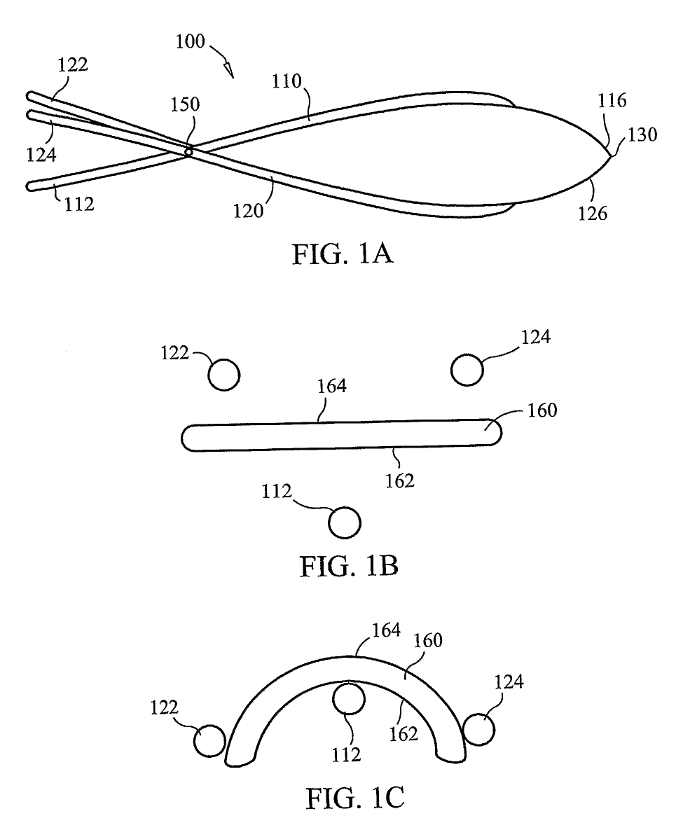

Referring first to FIG. 1A, there is illustrated a preferred embodiment of a forceps device for folding objects in accordance with the present invention, which has been designated generally by reference numeral 100. The forceps 100 include a first scissor arm 110 and a second scissor arm 120. The first scissor arm 110 has a first prong 112 formed at an end thereof and a handle 116 formed at a second end thereof.

The second scissor arm 120 has first and second spaced-apart prongs, 122 and 124, respectively, formed at an end thereof and includes a handle 126 formed at a second end thereof. Accordingly, forceps device 100 comprises three prongs. In the embodiment 100 shown, handles 116 and 126 are joined at point 130. In other embodiments of the inventive 3-prong forceps, handles 116 and 126 are not joined together at their ends.

As shown in FIG. 1A, the first scissor arm 110 is rotatably mounted to the second scissor arm 120 at mechanical joint 150.

FIGS. 1B and 1C illustrate a representative method for grasping and folding an object 160 with the forceps device 100, as viewed from the ends of the prongs. In FIG. 1B, the forceps are in the “open” position. The handles of the first and second scissor arms are spaced apart and the prong 112 formed on the first scissor arm is positioned below the object 160 and the first and second prongs 122/124 of the second scissor arm are positioned above the object 160. In the fully opened position, prongs 122/124 are positioned superior to prong 112 when viewed end-on.

Referring now to FIGS. 1A-C, when the spacing between the arms 110 and 120 is reduced, the prongs 122/124 and 112 begin to articulate about joint 150, and change position relative to one another. In the process of closing about object 160, prong 112 contacts the inferior surface 162 of object 160, and prongs 112 and 124 contact the superior surface 164 of object 160. In the “closed” position, the spacing between the first and second scissor arms 110/120 is further reduced and the prong 112 assumes a position superior to that of prongs 122/124, creating a fold thereby in object 160 (FIG. 1C). Thus folded, the object 160 is reduced in width, and can be removed from a smaller opening than would otherwise be possible in an unfolded state. As further described below, the device 100 can be used in conjunction with a tubular body (explantation device) for containing objects grasped and folded by the device 100.

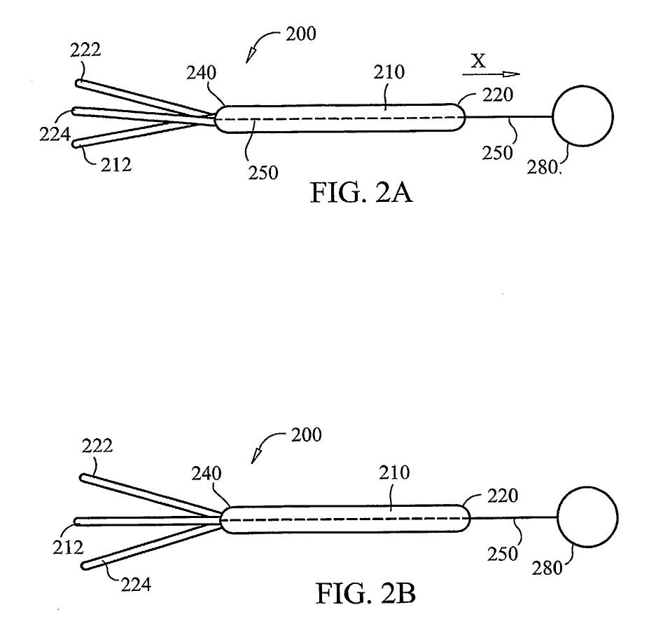

Referring now to FIG. 2A, there is illustrated a preferred embodiment of a retractable forceps device for grasping and folding objects in accordance with the present invention, which has been designated generally by reference numeral 200. Forceps device 200 includes, among other elements, a tubular main body 210 which has a proximal end 220 and a distal end 240.

The tubular main body 210 defines an interior object-receiving chamber which extends between the proximal and distal ends 220/240 and establishes the longitudinal axis “X” for device 200. The cross-section of the tubular main body 210, when viewed in a plane that is orthogonal to axis “X”, is circular or elliptical.

A longitudinal member 250 comprising several parts is contained within the object-receiving chamber, and extends beyond both ends 220/240 of the tubular main body 210. At its proximal end, the longitudinal member 250 is continuous with a handle 280, which may be in the form of a rounded knob, as shown in FIG. 2A, or a pull-ring. At its distal end, the longitudinal member 250 is operably connected to a forceps comprising a pair of prongs 222/224 and a single prong 212. As shown in FIG. 2A, when viewed from the side in the “open” position, paired prongs 222 and 224 are angled upward, such that their distal ends are on a plane superior to the longitudinal axis of longitudinal member 250, whereas single prong 212 is angled downward relative to the long axis of member 250. Viewed from above, prong 212 is seen to approximately bisect the angle created by the paired prongs 222/224 (FIG. 2B).

In order to close the prongs 222/224 and 212 of the retractable forceps device 200, a pulling force is applied on the handle 280 in the direction of the arrow indicated by “X” in FIG. 2A. As the longitudinal member 250 is pulled proximally toward the user, the prongs 222/224 and 212 are drawn into the receiving chamber 210, and the spacing between the prongs is reduced, as further described below, such that they can fit inside the chamber 210.

The components of the forceps devices can be made from a variety of biocompatible materials suitable for construction of surgical instruments, such as metal (e.g., surgical steel or titanium) or a synthetic material such as acrylonitrile butadiene styrene (ABS), liquid crystal polymer, (LCP), or polyetheretherketone (POLY). These materials are considered representative and are not intended to limit the scope of the available materials.

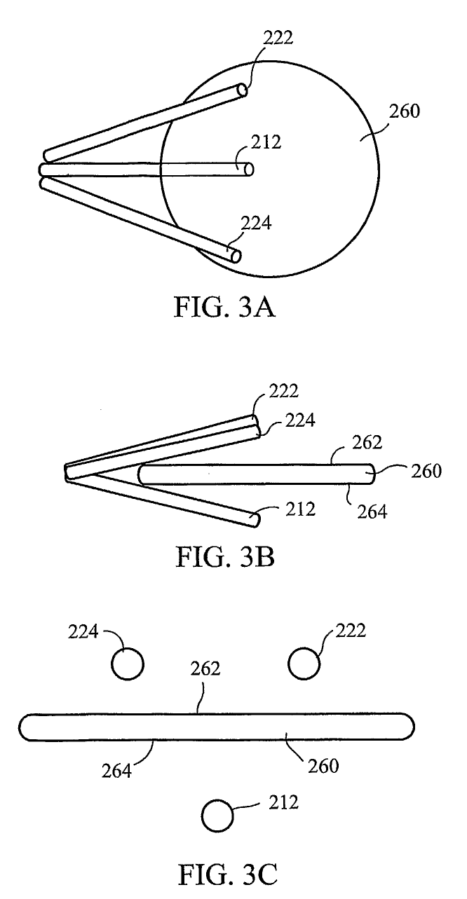

FIGS. 3-5 schematically illustrate a series of steps in the operation of a retractable forceps device 200 that are used to grasp and fold an object 260. To aid understanding, top, side and end-on views of corresponding structures are shown in the drawings. Referring now to FIGS. 3A and 3B, an object 260 is approached from the side with the device in the open position, with prongs 222 and 224 positioned relative to one surface of the object 260 (such as above its upper surface 262), and prong 212 positioned below the lower surface 264 of the object 260 (illustrated in FIGS. 3B and 3C). As shown in FIG. 3B, the object is grasped between the prongs by contacting the prongs 222/224 and 212 with the upper and lower surfaces of the object 260. As discussed above, such contact is achieved by pulling the handle 280, to exert backward force on the prongs by means of longitudinal member 250, drawing the prongs into the receiving chamber 210.

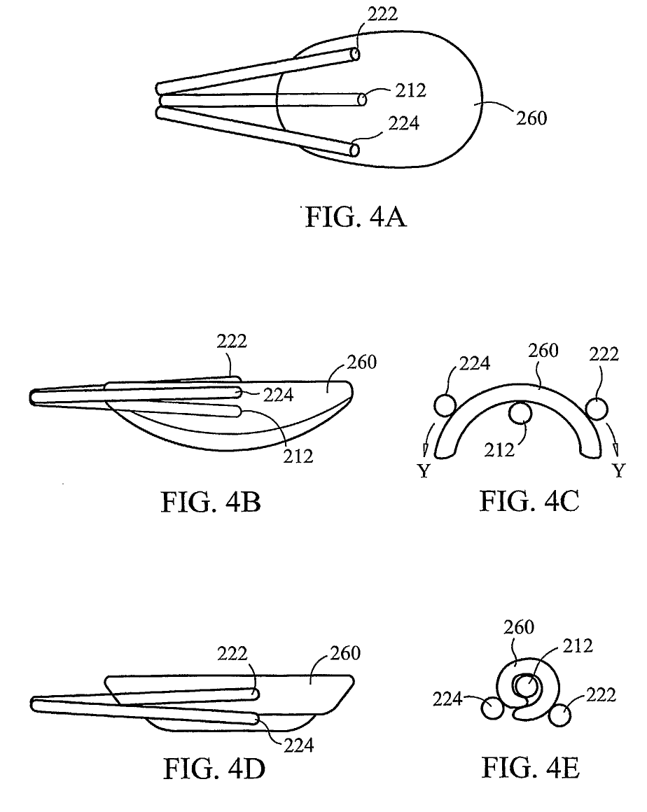

In the operation of the retractable forceps 200, folding of the object 260 occurs as the proximal portions of the prongs 222/224 and 212 are drawn into the object-receiving chamber 210. FIG. 4 illustrates successive steps in the folding process, as seen in top view (FIG. 4A; compare with FIG. 3A), in side view (FIGS. 4B, 4D) and as viewed end-on (with FIG. 4C corresponding to FIG. 4B, and FIG. 4E corresponding to FIG. 4D).

Referring now to FIG. 4, and by comparing with FIG. 3, it will be appreciated that folding of the object is achieved by two different types of movements of the prongs relative to one another, as the device is retracted and the prongs transition from the opened to closed position. One movement is upward displacement of the prong 212 positioned beneath the object 260, coupled with downward movement of the prongs 222 and 224 positioned above the object to create an initial fold, similar to the movement as described above for device 100. These movements may be further appreciated by comparing the side views presented in FIGS. 3B and 4B, and the end-on views presented in FIGS. 3C and 4C.

Referring again to FIG. 4, the second folding motion, which results in curling of the free edges of the object 260, is achieved by inward displacement and downward movement of the two prongs 222 and 224 positioned on the upper surface of the object, in the direction of the arrows indicated by “Y” in FIG. 4C. This movement reduces the angle between the prongs 222 and 224, as seen from above (compare FIGS. 3A and 4A). As the prongs move downward over the surface of the object in the direction of the arrows indicated by “Y” in FIG. 4C, this movement has the effect of curling the ends of the object upon itself in a jellyroll fashion (FIGS. 4D, E). With continued retraction of longitudinal member 250, the folded object 260 is drawn into the receiving chamber 210.

Those of skill in the art will appreciate that there are many potential surgical applications of the inventive forceps devices. As a particular non-limiting example in the field of ophthalmic surgery, a device and method useful for explantation of an intraocular lens (IOL) is now described.

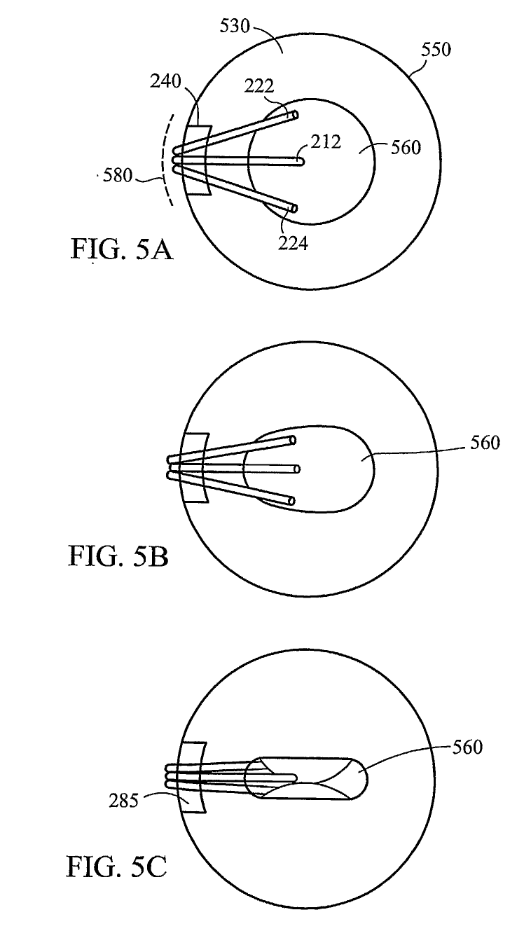

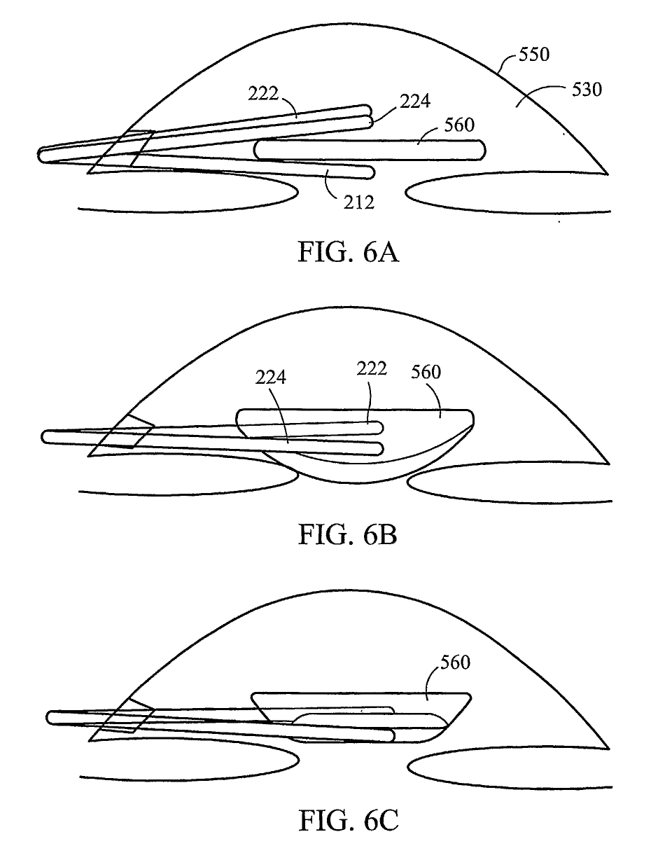

Depending on factors, such as for example, wound size and lens characteristics (e.g., stiffness), a retractable forceps 200 may be used exclusively to extract the IOL from the patient's eye. FIG. 5 illustrates successive steps (A-C) in a procedure utilizing the device 200 to grasp and fold an IOL 560 to be removed from the anterior chamber 530 of the eye of a patient, as seen from above (i.e., looking through the cornea 550). FIG. 6 schematically illustrates the same steps as viewed from the side.

In a representative application in which the IOL explantation device 200 is used to remove an artificial lens having a 6.0 mm optic, an elliptical cross-section for the tubular main body 210 preferably has a major axis “Y” length of about 2.7 mm and a minor axis “Z” length of about 1.65 mm. Configuring device 200 in this manner allows the distal end 240 of the device to fit within the original 3.0 mm wound 580 formed in the patient's cornea/limbus, as schematically illustrated in FIGS. 5A-C. This configuration allows the folded lens to be accommodated within the interior lens-receiving chamber 285 of the device 200. (See FIG. 5C).

Steps in the procedure for folding the lens within the anterior chamber of the eye are carried out by the surgeon essentially as described above, while viewing the prongs and the IOL 560 through the cornea 550 (FIGS. 5,6). Upon completion of the folding step, IOL 560 is withdrawn completely into the lens receiving chamber 285 and the device 200 is withdrawn through the incision site 580.

Alternatively, scissor arm forceps 100 as described above can be used in conjunction with a separate explantation device to impart a fold to the lens prior to retracting the lens into the interior chamber of the explantation device.

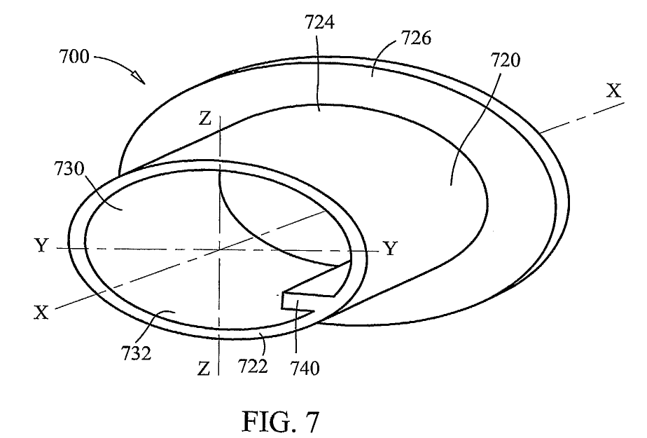

Referring now to FIG. 7, a representative explantation device 700 configured for lens explantation surgery comprises, among other elements, a tubular main body 720 which has a distal end 722 and a proximal end 724. The tubular main body 720 can be made from a variety of materials, such as acrylonitrile butadiene styrene (ABS), liquid crystal polymer, (LCP), or polyetheretherketone (POLY). These materials are considered representative and are not intended to limit the scope of the available materials. In a preferred embodiment, the tubular main body is formed from ABS due to its ability to be easily processed by injection or extrusion molding, and also its high chemical resistance and dimensional stability.

The tubular body 720 defines an interior lens-receiving chamber 730 which extends between the proximal and distal ends 722/724 and establishes the central axis “X” for the device 700. The cross-section of the tubular body 720, when viewed in a plane that is orthogonal to the central axis “X”, is elliptical and has a major axis “Y” and a minor axis “Z”.

In some embodiments, the tubular body of the present disclosure further includes a fin element 740. Fin element 740 is attached to inner surface 732 of tubular body 720 and projects inward from the inner surface 732 into the interior object receiving chamber 730. As shown in FIG. 7, the fin element 740 is attached to the inner surface 732 of the tubular main body 720 at a location offset from the major axis “Y” of the tubular main body 732, approximately 35 degrees below. The fin element 740 projects inward from inner surface 732 of tubular main body 720 in a plane that is parallel to major axis “Y” of main body 720. In a representative construction, the fin 740 projects inwardly approximately 0.5 mm from the inner surface 732 of the tubular main body 720.

Those skilled in the art will readily appreciate that the fin element 740 can be eliminated and a groove or channel can be formed in the inner surface 732 of the tubular main body 720 without departing from the inventive aspects of the present disclosure. Preferably, the groove or channel is located in a similar manner as described above for the fin element 740.

Preferably, the remainder of the inner surface of the tubular main body is smooth and free of protuberances. The inner surface should have a minimal coefficient of sliding friction to reduce any drag imparted on the lens during retraction into the lens-receiving chamber. Applicants note that helon gel can be applied to the inner surface of the main body to reduce the sliding friction between this surface and an intraocular lens.

Explantation device 700 further includes a stop flange 726 formed on the proximal end 724 of the tubular main body 720 (FIG. 7). Stop flange 726 prevents the explantation device 700 from being inserted too far into a patient's eye and also provides a means for a surgeon to hold the device during the procedure.

In a representative application in which the explantation device 700 is used, for example in conjunction with forceps 100 to remove an artificial lens having a 6.0 mm optic, the elliptical cross-section for the tubular main body 720 preferably has a major axis “Y” length of about 2.7 mm and a minor axis “Z” length of about 1.65 mm. Configuring explantation device 700 in this manner allows the device to fit within the original 3.0 mm wound formed in the patient's cornea, as discussed above.

A representative method for using forceps 100 in conjunction with explantation device 700 to remove an IOL after it has been positioned within the anterior chamber of the eye can be described as follows. A typical anterior chamber of a person's eye is 12 mm across and 4 to 5 mm deep in the center and becomes shallower toward the periphery.

To remove an IOL using forceps 100 in conjunction with lens explantation device 700, the distal end of explantation device 700 is positioned within a wound formed in the cornea. Once the explantation device 700 is properly positioned, forceps 100 are inserted into the proximal end of device 700 and are advanced through the interior of the device 700 beyond its distal end, into the anterior chamber of the eye. Forceps 100 are used to grasp the IOL and induce a fold in it, as described above. Forceps 100 are then retracted, drawing the intraocular lens into the interior lens-receiving chamber of the tubular main body of the explantation device 700.

Initially, the lens contacts the fin element 740 and a portion of the inner wall of the tubular main body and is partially restrained thereby. The lens is further retracted in the proximal direction by forceps 100 and fin element 740 continues to restrain a first edge of the lens. As the lens moves proximally, a second edge of the lens follows the curved inner surface of the tubular member and is folded or coiled thereby. Lastly, explantation device 700 which has the intraocular lens disposed within the interior lens receiving chamber is removed from the patient's eye.

All of the surfaces of explantation device 700 are preferably free of sharp edges, so as to reduce the potential for damage to the person's eye during surgery. As with any surgical tool, explantation device 700 is made from a biocompatible material, such as a polymer or a stainless steel. Moreover, those skilled in the art would readily appreciate that a lubricant can be used, if needed, to facilitate the sliding of the lens around the inner wall of the tubular body, and the folding of the IOL.

Applicants note that although explantation device 700 has been described above as including a fin element or groove/channel which facilitates the folding of the IOL, those skilled in the art would readily appreciate that removal of an IOL that is soft and flexible when implanted can be performed using a main tubular body that does not include fin element or have groove/channel formed in an inner surface thereof.

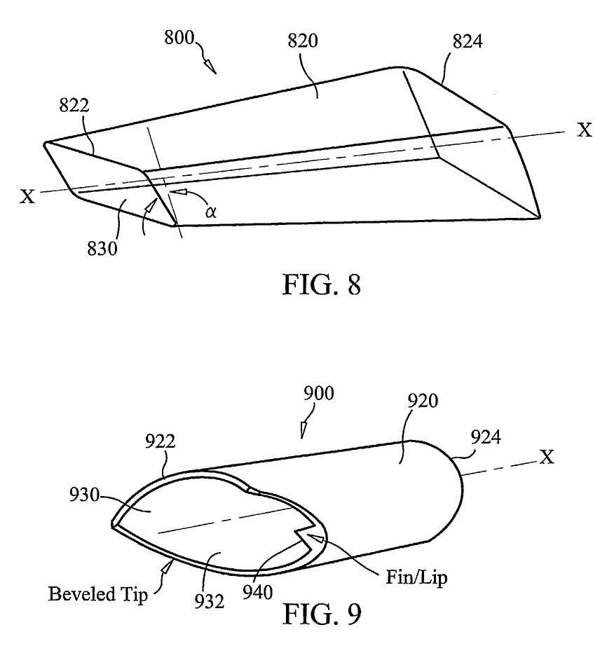

Referring now to FIG. 8, there is illustrated an alternative embodiment of the intraocular lens explantation device 800 of the present invention. Lens explantation device 800 is similar in construction and function to explantation device 700.

Like lens explantation device 700, lens explantation device 800 includes, among other elements, a tubular main body 820 which has a proximal end 824 and a distal end 822. The tubular main body 820 defines an interior lens receiving chamber 830 which extends between the distal and proximal ends 822/824 and establishes the central axis “X” for explantation device 800. However, unlike explantation device 700, the cross-section of the tubular main body 820, when viewed in a plane that is orthogonal to the central axis “X”, is rectangular.

The distal end 822 of the tubular main body 820 is beveled at an angle “α” to facilitate placement of the distal end within the wound of a patient's eye and to accommodate the natural curvature of the eye. Further, as can be readily ascertained from FIG. 8, the internal corners of the tubular main body 820 are rounded to prevent the edges of a retracted IOL from becoming captured therein.

Referring now to FIG. 9, there is illustrated an alternative embodiment of the intraocular lens explantation device of the present invention which has been designated generally by reference numeral 900. Lens explantation device 900 is similar in construction and use to explantation devices 700 and 800.

Like explantation device 700 and 800, lens explantation device 900 includes, among other elements, a tubular main body 920 which has a proximal end 924 and a distal end 922. The tubular main body 920 defines an interior lens-receiving chamber 930 which extends between the distal and proximal ends 922/924 and establishes the central axis “X” for explantation device 900.

Explantation device 900 also includes a fin element 940. Fin element 940 is attached to inner surface 932 of tubular main body 920 and projects inward from the inner surface 932 into the interior lens receiving chamber 930. As shown in FIG. 9, the fin element 940 is attached to the inner surface 932 of the tubular main body 920. However, unlike fin element 740 used with explantation device 700, fin element 940 is not substantially rectangular in cross-section, but is a rounded protuberance.

### Alternative Embodiments of the Lens Explantation Device

Provided below are descriptions for additional alternative devices which can be used for IOL explantation.

One alternative embodiment includes a three-pronged forceps device. In this design, three prongs originate from a plastic tube having a preferably 2.7 mm diameter. Two axially movable prongs are adapted and configured for extending over the top of the lens while a stationary third prong is positioned beneath the lens. While taking precaution to avoid cornea abrasion by the forceps device, the two axially movable prongs are retracted over the third prong, causing the lens to fold neatly in half.

A further device for lens explantation includes a probe with suction element. The end of the probe is placed in contact with the lens surface, and then a button located on the tool itself is actuated to begin the suction. The lens is then pulled into a cannula having dimensions small enough to meet the 3.0 mm wound size.

A further embodiment includes a cylindrical-shaped tool which is used to curl the lens asymmetrically. A curved cylinder is inserted beneath the intraocular lens. A moon-shaped gripping device approaches the lens from above. At the control of the surgeon, the two pieces would squeeze together causing the lens to curl around the bottom cylinder. The curling affect creates a smaller cross-sectional diameter and allows the lens to be removed through the 3.0 mm cannula.

A yet further device includes a tube having an internal helical track formed on its inner surface. The interior of the tube is lined with a silicone gel to maximize surface tension to prevent the lens from returning to the eye cavity once inside the tube. The track in which the lens traverses runs though a helical pattern, which leads the lens to fold into an “s” shape manner such that the lens reaches a sub-3.0 mm profile once it reaches the end of its travel out of the anterior chamber.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A forceps device for folding an object comprising:
a) a first scissor arm having a first prong formed at an end thereof and a handle formed at a second end thereof; and
b) a second scissor arm having first and second spaced-apart prongs formed at an end thereof and including a handle formed at a second end thereof, the second scissor arm being rotatably mounted to the first scissor arm; and
wherein in the open position the handle portions of the first and second scissor arms are spaced apart and the first prong formed on the first scissor arm is positioned below the first and second prongs of the second scissor arm; and
wherein in the closed position the spacing between the handle portions of the first and second scissor arms is reduced and the first prong formed on the first scissor arm is positioned above the first and second prongs of the second scissor arm, and wherein a fold is imparted in an object contacted by said prongs during movement from the open position to the closed position.

2. A retractable forceps device for folding and containing an object comprising:
a) a tubular body member having a proximal end and a distal end and an inner surface extending therebetween;
b) a first arm having a first prong formed at an end thereof and a handle formed at a second end thereof; and
b) a second arm having first and second spaced-apart prongs formed at an end thereof and a handle formed at a second end thereof, said first and second arms and prongs being operatively connected to said handle and retractable into said tubular body member by means of said handle; and
wherein in the open position the prongs are extended distal to the tubular body member and the first prong formed on the first arm is positioned below the first and second prongs of the second arm; and
wherein in the closed position at least the proximal portion of the prongs is retracted into the tubular body member and the first prong formed on the first arm is positioned above the first and second prongs of the second arm, and the angle between the first and second prongs on the second arm is reduced, wherein a fold is imparted in an object contacted by said prongs during movement of the prongs from the opened to the closed position, said movement being effectuated by retracting said prongs into said tubular body member.

3. A retractable forceps device as recited in claim 2 for use in explanting an intraocular lens from a patient's eye, the device comprising a tubular body member having a proximal end and a distal end and an inner surface extending therebetween, the inner surface defining an interior lens-receiving chamber adapted and configured for receiving an intraocular lens.

4. The device as recited in claim 3, wherein the cross-section of the tubular body member taken in a plane which is orthogonal to the central axis is elliptical.

5. The device as recited in claim 4, wherein the elliptical cross-section for the tubular body member has a major axis of about 2.7 mm and a minor axis of about 1.65 mm.

6. A device for folding and containing an object, the device comprising the forceps device as recited in claim 1 and a tubular device for guiding the forceps and containing the folded object.

7. A device according to claim 6 for use in explanting an intraocular lens from a patient's eye, wherein the tubular device is a lens explantation device comprising a tubular main body having a proximal end and a distal end and an inner surface extending therebetween, the inner surface defining an interior lens-receiving chamber and a central axis for the explantation device.

8. The device as recited in claim 7, wherein the cross-section of the tubular main body taken in a plane which is orthogonal to the central axis is elliptical.

9. The device as recited in claim 8, wherein the elliptical cross-section for the tubular main body has a major axis of about 2.7 mm and a minor axis of about 1.65 mm.

10. The device as recited in claim 9, wherein the inner surface of the tubular main body has an axial groove formed therein which is adapted and configured for receiving an edge of an intraocular lens.

11. The device as recited in claim 10, wherein the groove is formed in the inner surface of the tubular main body at a location offset from the major axis of the tubular main body.

12. The device as recited in claim 11, wherein the groove is formed in the inner surface of the tubular main body at a location about 35 degrees below the major axis of the tubular main body.

13. The device as recited in claim 11, wherein the groove is formed in the inner surface of the tubular main body in a plane parallel to the major axis of the tubular main body.

14. The device as recited in claim 10, wherein the groove has a depth of approximately between and including 0.25 to about 0.5 mm from the inner surface of the tubular main body.

15. The device as recited in claim 7, further comprising a stop flange associated with the proximal end of the tubular main body for preventing over-insertion of the explantation device into a patient's eye.

16. The device as recited in claim 7, wherein the proximal end of the tubular main body includes a beveled tip portion to facilitate safe placement of the tubular main body into the wound formed in a patient's eye and receiving the lens into the interior lens-receiving chamber.

17. A method for explanting an intraocular lens from the anterior chamber of a patient's eye, comprising the steps of:
a) providing a retractable forceps device comprising a tubular body member having a proximal end and a distal end and an inner surface extending therebetween, a first arm having a first prong formed at an end thereof and a handle formed at a second end thereof and a second arm having first and second spaced-apart prongs formed at an end thereof and a handle formed at a second end thereof, said first and second arms and prongs being operatively connected to said handle and retractable into said tubular body member by means of said handle, wherein in the open position the prongs are positioned beyond the distal end of the tubular body member and the first prong formed on the first arm is positioned below the first and second prongs of the second arm, and wherein in the closed position at least the proximal portion of the prongs is retracted into the tubular body member and the first prong formed on the first arm is positioned above the first and second prongs of the second arm, and the angle between the first and second prongs on the second arm is reduced, wherein a fold is imparted in an object contacted by said prongs during movement of the prongs from the opened to the closed position, said movement being effectuated by retracting said prongs into said tubular body member;
b) inserting the distal end of the tubular body member into a wound formed in the patient's cornea;
c) advancing the prongs of the forceps device beyond the distal end of the tubular body member into the anterior chamber of the eye;
d) grasping and folding the intraocular lens with the prongs of the retractable forceps;
e) retracting the forceps, thereby withdrawing the grasped and folded lens into the interior lens-receiving chamber; and
f) removing the retractable forceps device having the intraocular lens disposed within the interior lens-receiving chamber from the patient's eye.

18. A method for explanting an intraocular lens from the anterior chamber of a patient's eye, comprising the steps of:
a) providing an explantation device comprising a tubular main body having a proximal end and a distal end and an inner surface extending therebetween, the inner surface defining an interior lens-receiving chamber and a central axis for the explantation device;
b) inserting the distal end of the explantation device into a wound formed in the patient's cornea;
c) inserting a means for grasping the intraocular lens into the interior lens-receiving chamber so that the lens-grasping means extends from the proximal end of the tubular main body beyond the distal end of the tubular main body into the anterior chamber of the eye;
d) grasping the intraocular lens with the grasping means;
e) retracting the lens into the interior lens-receiving chamber such that the lens is folded thereby; and
f) removing the explantation device having the intraocular lens disposed within the interior lens-receiving chamber from the patient's eye.

19. The method as recited in claim 18, further comprising the step of applying a lubricant to the inner surface of the tubular main body to reduce the sliding friction between the lens and the inner wall.

20. The method as recited in claim 18, further comprising the step of pre-folding the lens with the grasping means.

21. The method as recited in claim 20, wherein the grasping means comprises a forceps device for folding an object comprising a first scissor arm having a first prong formed at an end thereof and a handle formed at a second end thereof and a second scissor arm having first and second spaced apart prongs formed at an end thereof and including a handle formed at a second end thereof, the second scissor arm being rotatably mounted to the first scissor arm, and wherein in the open position the handle portions of the first and second scissor arms are spaced apart and the first prong formed on the first scissor arm is positioned below the first and second prongs of the second scissor arm and wherein in the closed position the spacing between the handle portions of the first and second scissor arms is reduced and the first prong formed on the first scissor arm is positioned above the first and second prongs of the second scissor arm, and wherein a fold is imparted in an object contacted by said prongs during movement from the open position to the closed position, the method further comprising positioning the forceps such that the lens is folded.
